(19) United States
(12) Patent Application Publication
(10) Pub. No.: US 2011/0130413 A1
(43) Pub. Date: Jun. 2, 2011

(54) SUBSTITUTED QUINAZOLINES

(75) Inventors: Bernard Golding, Tyne and Wear (GB); Peter Cicala, Wayne, PA (US); Richard Franklin, Hampshire (GB); Angus Macleod, Essex (GB)

(73) Assignee: Shire LLC, Florence, KY (US)

(21) Appl. No.: 12/992,990

(22) PCT Filed: May 13, 2009
(86) PCT No.: PCT/GB2009/050508

§ 371 Date: Feb. 14, 2011

(30) Foreign Application Priority Data
May 16, 2008 (GB) 0808948.4

(51) Int. Cl. A61K 31/517; C07D 239/84; A61P 7/02

(52) U.S. Cl. 514/266.4; 544/292

(57) ABSTRACT

This invention relates to the discovery of prodrugs of substituted analogues of the selective platelet lowering agent anagrelide which have reduced potential for cardiovascular side-effects and which should therefore lead to improved patient compliance and safety in the treatment of myeloproliferative diseases. More specifically, the present invention relates to prodrugs of certain imidazoquinazoline derivatives which have the general formula (I) shown below wherein the substituents have the meanings defined in claim 1 and which have utility as platelet lowering agents in humans. The compounds of the present invention function by inhibiting the formation of blood platelets.

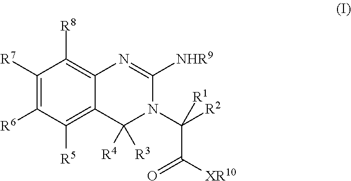

### FIELD OF THE INVENTION

This invention relates to the discovery of prodrugs of substituted analogues of the selective platelet lowering agent anagrelide which have reduced potential for cardiovascular side-effects and which should therefore lead to improved patient compliance and safety in the treatment of myeloproliferative diseases. More specifically, the present invention relates to prodrugs of certain imidazoquinazoline derivatives which have utility as platelet lowering agents in humans. The compounds of the present invention function by inhibiting the formation of blood platelets.

### BACKGROUND OF THE INVENTION

Anagrelide hydrochloride (Agrylin®, Xagrid®) is a novel orally administered imidazoquinazoline which selectively reduces platelet count in humans and is used for such purposes in the treatment of myeloproliferative diseases (MPDs), such as essential thrombocythemia (ET), where an elevated platelet count may put the patient at increased thrombotic risk. The chemical structure of anagrelide, 6,7-dichloro-1,5-dihydroimidazo[2,1-b]-quinazolin-2(3H)-one (hydrochloride monohydrate), is shown in the following formula:

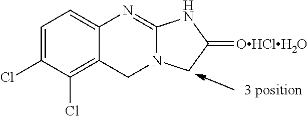

The principal side effects of anagrelide are cardiovascular in nature, tachycardia, palpitations etc and limit the utility of the drug. These are largely attributed to its metabolism to 3-hydroxyanagrelide. This compound was surprisingly found to be some 40-fold more potent as an inhibitor of PDEIII, and therefore potential inotropic agent, than anagrelide itself. Furthermore plasma exposure to this metabolite after treatment with anagrelide is typically three times greater than to the drug itself confirming its pivotal role. Consequently a series of 3-substituted anagrelide analogues has been investigated which has shown that it is possible to introduce metabolism blocking groups at that position and yet still retain the anti-megakaryocytic actions of the drug. Furthermore, these compounds have considerably less potential for cardiovascular effects than 3-hydroxyanagrelide. Indirect steric hindrance to the formation of the 3-hydroxy metabolite may also be achieved by substitution at the 5-position. The 3-substituted compounds typified, by the dimethyl or spirocyclopropyl analogues, are notably less soluble at physiological pH (˜7) than the parent compound which presents a significant challenge to their efficient absorption.

Anagrelide HCl itself is a poorly soluble drug substance. In the pH range of 4-8, the solubility is less than 10 μg/mL. The solubility increases at pH values above and below this range; for example in 0.1M HCl the solubility is ˜170 μg/mL and at pH 11.4 approaches 1 mg/mL. The dissociation constants (pKa1 and pKa2) of 2.9 and 9.8 were estimated from the solubility/pH profile of anagrelide HCl. Thus over much of the physiological pH range the drug has very poor aqueous solubility. Even material dissolving in the stomach at pH 1-2 may precipitate in the duodenum at pH 5-6. This presents potential problems for the quantitative absorption from the most likely site for absorption, namely the upper small intestine. As a consequence anagrelide is micronised prior to filling into capsules for clinical use to ensure maximal absorption. This and anagrelide's inherent potency as an anti-megakaryocytic agent—in vitro IC₅₀˜27 nM and in vivo doses of just 1-2 mg—serve to limit the potential problem of incomplete absorption. Indeed, a radiolabelling study in humans showed that following the oral administration of 1 mg ¹⁴C-labelled drug >75% of the administered radioactivity was recovered in the urine implying that at least after this dose absorption was >75%. However for those patients requiring larger doses either as the result of relative insensitivity to the drug, higher first-pass pre-systemic metabolism or greater body weight, the possibility exists for incomplete absorption. This would be expected to lead to increased variability in attained plasma drug concentrations and consequential variability in patient response.

Furthermore for less potent analogues of anagrelide, even though they may have better cardiovascular profiles, problems of incomplete absorption may be encountered. Improved water solubility through the use of appropriate open ring pro-drugs of anagrelide may therefore offer significant advantage in minimizing this risk.

WO2004/063172 relates to the use of 2-amino-2H-quinazoline derivatives for producing therapeutic agents for the treatment of myeloproliferative diseases, high blood pressure and bronchodilation.

### SUMMARY OF THE INVENTION

This invention provides for prodrugs of anagrelide derivatives substituted at either the 3- or 5-position. In these anagrelide derivatives, metabolism to an analogue of the cardioactive 3-hydroxyanagrelide is blocked either directly (3-substitution) or indirectly (5-substitution). The prodrugs are notably more soluble in vitro (and under anticipated in vivo conditions) than their ring closed analogues offering the potential for better absorption from the GI tract. Such compounds would spontaneously and completely ring close at pH 7 or above thus offering a convenient means of delivering these ring closed anti-megakaryocytic (platelet lowering) agents to the systemic circulation. Since the preferred site of metabolism of anagrelide is the 3-position, such compounds are likely to present improved pharmacokinetic profile and hence improve patient compliance and convenience enabling a broader spectrum of patients to be effectively treated. In the case of the 5-substituted derivatives it is expected that a bulky group is more effective than a smaller group when cyclised to the ‘closed ring’ anagrelide analogue. Groups such as t-butyl and other bulky blocking groups are thus expected to be of most utility when substituted at the 5-position. A substituent comprising a large group at the 5-position is expected to sterically hinder access to the 3-position by the metabolising cytochrome's active site. This should inhibit formation of the cardioactive metabolite, 3-hydroxyanagrelide.

The ring closed compounds of the present invention are especially beneficial because surprisingly they have dramatically lower PDE III inhibitory activity (and hence lower cardioactive potential) than the active metabolite of anagrelide, 3-hydroxyanagrelide and yet also surprisingly retain their anti-megakaryocytic activity. Indeed these compounds have therapeutic indices which are much more favourable than that for anagrelide itself.

In one embodiment, the present invention comprises a prodrug of an anagrelide analogue comprising a 3-, 5-, 3,3- or 5,5-substituted anagrelide compound. Thus, for example, in the 3-substituted derivatives, first pass metabolism (of the rapidly ring closed analogue) to 3-hydroxyanagrelide is directly blocked. In particular, the invention relates to prodrugs of an anagrelide analogue wherein first pass metabolism to the corresponding analogue of 3-hydroxyanagrelide is effectively blocked.

According to the present invention, there is provided a compound of Formula (I) or a pharmaceutically acceptable salt or solvate thereof:

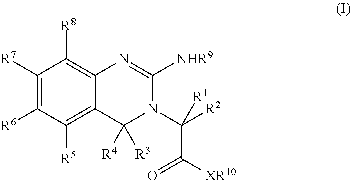

wherein:
R¹, R², R³ and R⁴ independently represent hydrogen or a blocking group which functions to prevent metabolic reaction either directly or indirectly at the carbon atom to which R¹ and R² are attached;
or R¹ and R², and/or R³ and R⁴ together with the carbon to which they are attached form a blocking group which functions to prevent metabolic reaction at the carbon atom to which R¹ and R² are attached, the remainder of groups R¹ to R⁴ being hydrogen;
R⁵, R⁶, R⁷ and R⁸ are each independently selected from hydrogen, R^{a} and R^{b};
R⁹ is H or C₁₋₆ alkyl;
R¹⁰ is selected from the group comprising: hydrogen; C₁₋₆ alkyl, C₂₋₆ alkenyl, C₂₋₆ alkynyl and C₃₋₈ cycloalkyl wherein each of the foregoing groups may be optionally substituted by 1 to 5 groups chosen independently from the group comprising: halo, hydroxyl, cyano, nitro, C₁₋₄ alkylsulphonyl and COOH; or R¹⁰ is a pharmaceutically acceptable cation;
X is O or S;
R^{a} is selected from C₁₋₆ alkyl and C₂₋₆ alkenyl, either of which is optionally substituted with 1, 2, 3, 4 or 5 R^{b};
R^{b} is selected from halo, trifluoromethyl, cyano, nitro, —OR^{c}, —C(O)R^{c}, —C(O)OR^{c}, —OC(O)R^{c}, —S(O)₁R^{c}, —N(R^{c})R^{d}, —C(O)N(R^{c})R^{d}, —N(R^{c})C(O)R^{d}, —S(O)₁N(R^{c})R^{d} and —N(R^{c})S(O)₁R^{d};
R^{c} and R^{d} are each independently hydrogen or R^{e};
R^{e} is selected from C₁₋₆ alkyl and C₂₋₆ alkenyl, either of which is optionally substituted with 1, 2, 3, 4 or 5 substituents independently selected from halo, cyano, amino, hydroxy, nitro and C₁₋₆ alkoxy; and
1 is 0, 1 or 2;
and wherein R¹, R², R³ and R⁴ are not all hydrogen.

In an embodiment when R⁵ and R⁶ are each halo, then R⁷ and R⁸ are not both selected from H, halo, cyano, C₁₋₆ alkyl, C₁₋₆ haloalkyl, C₁₋₆ alkoxy and C₁₋₆ haloalkoxy.

In an embodiment when one of R¹ and R² is methyl and R³ and R⁴ are hydrogen then the other of R¹ and R² is not hydrogen.

In an embodiment:

R¹ and R², are independently selected from the group comprising: H; halo; cyano; C₁₋₆ alkyl, C₂₋₆ alkenyl, C₂₋₆ alkynyl, C₃₋₈ cycloalkyl wherein said alkyl, alkenyl, alkynyl or cycloalkyl groups may be optionally substituted by 1 to 5 groups chosen independently from the group comprising: halo, hydroxyl, cyano, nitro, C₁₋₄ alkylsulphonyl and COOH; C₁₋₆ hydroxyalkyl; C₁₋₆ carboxyalkyl; and sulphide;
or R¹ and R² together with the carbon to which they are attached form a C₃₋₈ carbocyclic ring may be optionally substituted by 1 to 5 groups chosen independently from the group comprising: halo, hydroxyl, cyano, nitro, C₁₋₄ haloalkyl, C₁₋₄ alkylsulphonyl and COOH;
or R¹ and R² together with the carbon to which they are attached represent a C₂₋₆ alkenyl or C₂₋₆ alkynyl group bound through a double bond to the ring to which it is attached and being optionally substituted by one to three groups independently selected from the group comprising: halo, hydroxyl, cyano, C₁₋₄ haloalkyl and COOH, provided always that one of R¹ and R² is not hydroxyl when the other is methyl.

In a preferred set of compounds, R¹ is an optionally substituted C₁₋₄ alkyl or C₃₋₈ cycloalkyl group.

In a preferred set of compounds, R² is an optionally substituted C₁₋₄ alkyl or C₃₋₈ cycloalkyl group.

Other preferred compounds are those in which at least one of R¹ and R² is —C(H)ₙ(F)ₘ or —C(H)ₙ(F)ₘ—C(H)ₚ(F)_{q}, where m=2 or 3, and n=(3−m); and p=2 or 3, and q=(3−p).

More preferably at least one of R¹ and R² is CF₃ or CHF₂. Most preferably, at least one of R¹ and R² is CF₃.

In an embodiment, R¹ is preferably methyl, cyclopropyl, CF₃ or CHF₂. Most preferably, R¹ is methyl.

In an embodiment, R² is preferably methyl, cyclopropyl, CF₃ or CHF₂. Most preferably R² is methyl.

In another preferred set of compounds, R¹ and R² together form an optionally substituted C₃₋₈ cycloalkyl group. Most preferably this is a cyclopropyl group.

In a particular set of compounds, R¹ and R² are each methyl or together form methylene; or R¹ and R², taken together with the carbon atom to which they are attached, form cyclopropyl.

In an embodiment:

R³ and R⁴ are independently selected from the group comprising: H; halo; cyano; C₁₋₆ alkyl, C₂₋₆ alkenyl, C₂₋₆ alkynyl, C₃₋₈ cycloalkyl wherein said alkyl, alkenyl, alkynyl or cycloalkyl groups may be optionally substituted by 1 to 5 groups chosen independently from the group comprising: halo, hydroxyl, cyano, nitro, C₁₋₄ alkylsulphonyl and COOH; C₁₋₆ hydroxyalkyl; C₁₋₆ carboxyalkyl; and sulphide;
or R³ and R⁴ together with the carbon to which they are attached form a C₃₋₈ carbocyclic ring may be optionally substituted by 1 to 5 groups chosen independently from the group comprising: halo, hydroxyl, cyano, nitro, C₁₋₄ haloalkyl, C₁₋₄ alkylsulphonyl and COOH;
or R³ and R⁴ together represent a C₂₋₆ alkenyl or C₂₋₆ alkynyl group bound through a double bond to the ring to which it is attached and being optionally substituted by one to three groups independently selected from the group comprising: halo, hydroxyl, cyano, C₁₋₄ haloalkyl and COOH.

In an embodiment, R³ is H or C₁₋₆ alkyl. Preferably, R³ is H.

In an embodiment, R⁴ is H or C₁₋₆ alkyl. Preferably, R⁴ is H.

In an embodiment, R⁹ is H or Me. In one embodiment, R⁹ is H and compounds in which R⁹ is H enjoy good solubility. When R⁹ is a C₁₋₆ alkyl group, such as Me, the PDE III inhibiting activity is effectively eliminated. Me represents a particularly preferred alkyl substituent.

In an embodiment, R¹⁰ is H or optionally substituted C₁₋₆ alkyl. Most preferably, R¹⁰ is C₁₋₆ alkyl. In an alternative embodiment, R¹⁰ is Na or K, with Na being preferred.

In an embodiment, X is O.

In a further embodiment:

R¹ and R² are independently selected from the group comprising: H; cyano; C₁₋₆ alkyl, C₂₋₆ alkenyl, C₂₋₆ alkynyl, C₃₋₈ cycloalkyl wherein said alkyl, alkenyl, alkynyl or cycloalkyl groups may be optionally substituted by 1 to 5 groups chosen independently from the group comprising: halo, hydroxyl, cyano, nitro, C₁₋₄ alkylsulphonyl and COOH; C₁₋₆ hydroxyalkyl; C₁₋₆ carboxyalkyl; and sulphide;
or R¹ and R² together with the carbon to which they are attached form a C₃₋₈ carbocyclic ring may be optionally substituted by 1 to 5 groups chosen independently from the group comprising: halo, hydroxyl, cyano, nitro, C₁₋₄ haloalkyl, C₁₋₄ alkylsulphonyl and COOH;
or R¹ and R² together represent a C₂₋₆ alkenyl or C₂₋₆ alkynyl group bound through a double bond to the ring to which it is attached and being optionally substituted by one to three groups independently selected from the group comprising: halo, hydroxyl, cyano, C₁₋₄ haloalkyl and COOH;
R³ and R⁴ are hydrogen; and
R⁷, R⁸, R⁹ and R¹⁰ are hydrogen.

Another preferred group of compounds is those in which neither R¹ nor R² is hydrogen. Amongst these, it is preferred when R¹ and R² are both independently selected from the group comprising: cyano, C₁₋₆ alkyl, C₂₋₆ alkenyl, C₂₋₆ alkynyl, in which the alkyl, alkenyl, and alkynyl groups may be optionally substituted;

or wherein R₁ and R₂ together with the carbon to which they are attached form an optionally substituted C₃₋₈ carbocyclic ring
or wherein R₁ and R₂ together represent an optionally substituted C₂₋₆ alkenyl or C₂₋₆ alkynyl group.

In an embodiment R^{a} is C₁₋₆ alkyl optionally substituted with 1, 2, 3, 4 or 5 R^{b}.

In an embodiment R^{a} is C₁, C₂, C₃ or C₄ alkyl, any of which is optionally substituted with 1, 2 or 3 R^{b}.

In an embodiment R^{b} is selected from halo, trifluoromethyl, cyano, nitro, —OR^{c}, —C(O)R^{c}, —C(O)OR^{c}, —OC(O)R^{c}, —S(O)₁R^{c}, —N(R^{c})R^{d}, —C(O)N(R^{c})R^{d}, —N(R^{c})C(O)R^{d}, —S(O)₁N(R^{c})C(O)R^{d} and —N(R^{c})S(O)₁R^{d}; wherein R^{c} and R^{d} are each independently hydrogen or C₁₋₆ alkyl optionally substituted with 1, 2, 3, 4 or 5 substituents independently selected from halo, cyano, amino, hydroxy, nitro and C₁₋₆ alkoxy.

In an embodiment R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy.

In an embodiment, R⁵, R⁶, R⁷ and R⁸ are each hydrogen.

In an embodiment the compound is of one of the following Formulae:

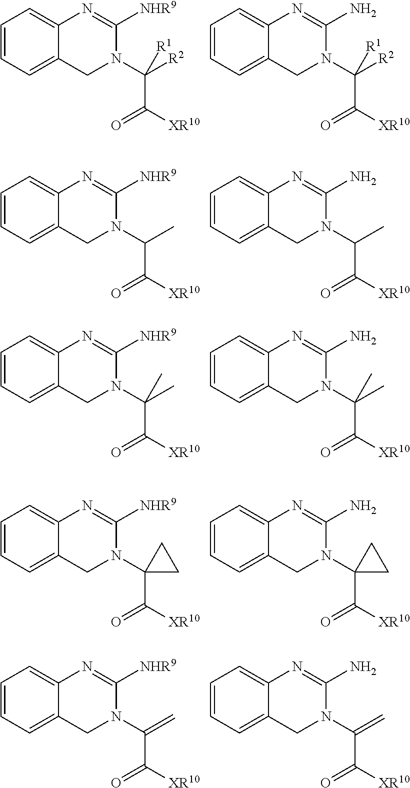

or, in each case, a pharmaceutically acceptable salt or solvate thereof.

In an embodiment, three of R⁵, R⁶, R⁷ and R⁸ are hydrogen, and the other is selected from R^{a} and R^{b}. Of mention are compounds in which R⁷ and R⁸ are each hydrogen.

In an embodiment the compound is of one of the following Formulae:

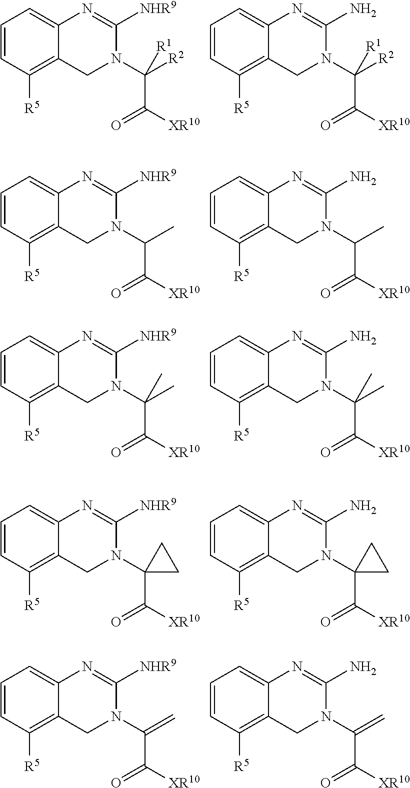

wherein R⁵ is selected from R^{a} and R^{b};
   or, in each case, a pharmaceutically acceptable salt or solvate thereof.

With regard to each of the above Formulae, R⁵ may be, for example, selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{C}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁵ is selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment the compound is of one of the following Formulae:

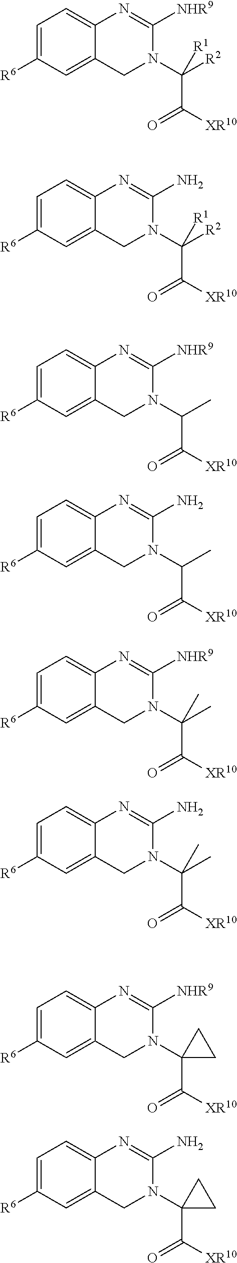

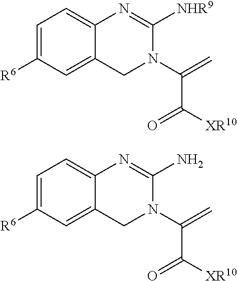

wherein R⁶ is selected from R^{a} and R^{b};
   or, in each case, a pharmaceutically acceptable salt or solvate thereof.

With regard to each of the above Formulae, R⁶ may be, for example, selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)O R^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁶ is selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment the compound is of one of the following Formulae:

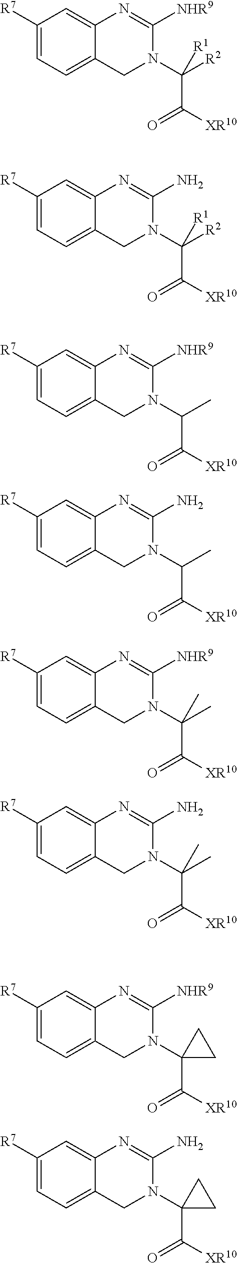

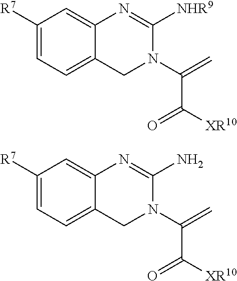

wherein R⁷ is selected from R^{a} and R^{b};
   or, in each case, a pharmaceutically acceptable salt or solvate thereof.

With regard to each of the above Formulae, R⁷ may be, for example, selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁷ is selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment the compound is of one of the following Formulae:

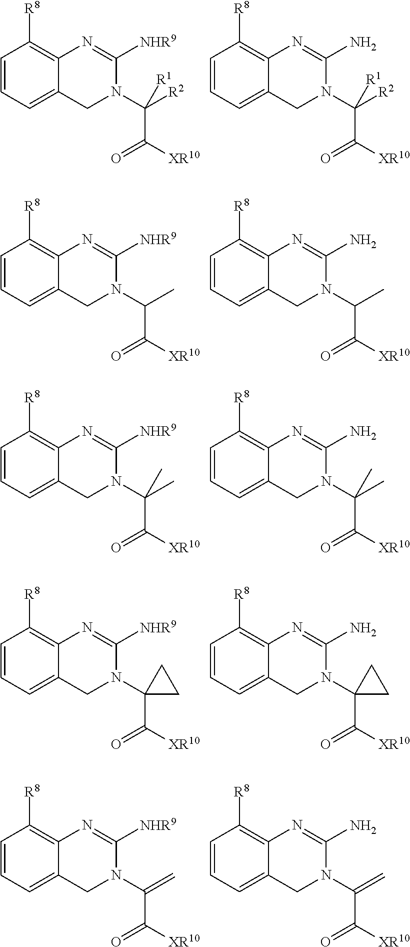

wherein R⁸ is selected from R^{a} and R^{b};
   or, in each case, a pharmaceutically acceptable salt or solvate thereof.

With regard to each of the above Formulae, R⁸ may be, for example, selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c}) R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁸ is selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment, two of R⁵, R⁶, R⁷ and R⁸ are hydrogen, and the other two are independently selected from R^{a} and R^{b}. Of mention are compounds in which R⁷ and R⁸ are each hydrogen.

In an embodiment the compound is of one of the following Formulae:

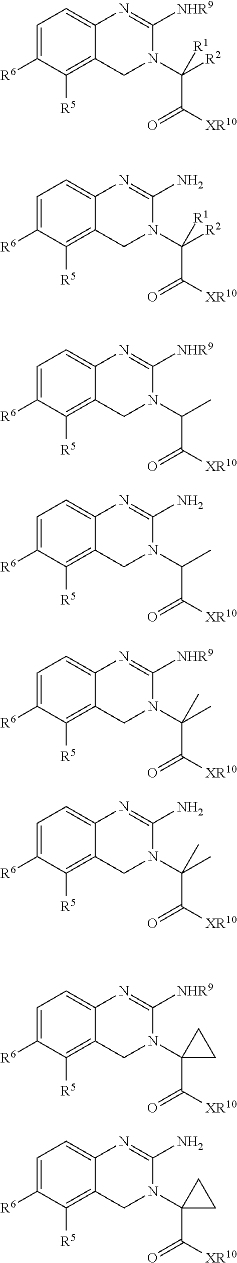

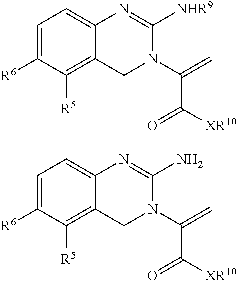

wherein R⁵ and R⁶ are each independently selected from R^{a} and R^{b};
   or, in each case, a pharmaceutically acceptable salt or solvate thereof.

With regard to each of said Formulae, R⁵ and R⁶ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁵ and R⁶ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment, R⁵ is R^{f} and R⁶ is R^{g}, wherein R^{f} and R^{g} are as defined in the Table below:

| R^{f} | R^{g} |
|---|---|
| —CN | Halo |
| —OH | Halo |
| —N(O)₂ | Halo |
| C₁₋₆ alkyl | Halo |
| C₁₋₆ alkoxy | Halo |
| —C(O)OH | Halo |
| —S(O)₂—C₁₋₆ alkyl | Halo |
| Halo | —CN |
| —CN | —CN |
| —OH | —CN |
| —N(O)₂ | —CN |
| C₁₋₆ alkyl | —CN |
| C₁₋₆ alkoxy | —CN |
| —C(O)OH | —CN |
| —S(O)₂—C₁₋₆ alkyl | —CN |
| Halo | —OH |
| —CN | —OH |
| —OH | —OH |
| —N(O)₂ | —OH |
| C₁₋₆ alkyl | —OH |
| C₁₋₆ alkoxy | —OH |
| —C(O)OH | —OH |
| —S(O)₂—C₁₋₆ alkyl | —OH |
| Halo | —N(O)₂ |
| —CN | —N(O)₂ |
| —OH | —N(O)₂ |
| —N(O)₂ | —N(O)₂ |
| C₁₋₆ alkyl | —N(O)₂ |
| C₁₋₆ alkoxy | —N(O)₂ |
| —C(O)OH | —N(O)₂ |
| —S(O)₂—C₁₋₆ alkyl | —N(O)₂ |
| Halo | C₁₋₆ alkyl |
| —CN | C₁₋₆ alkyl |
| —OH | C₁₋₆ alkyl |
| —N(O)₂ | C₁₋₆ alkyl |
| C₁₋₆ alkyl | C₁₋₆ alkyl |
| C₁₋₆ alkoxy | C₁₋₆ alkyl |
| —C(O)OH | C₁₋₆ alkyl |
| —S(O)₂—C₁₋₆ alkyl | C₁₋₆ alkyl |
| Halo | C₁₋₆ alkoxy |
| —CN | C₁₋₆ alkoxy |
| —OH | C₁₋₆ alkoxy |
| —N(O)₂ | C₁₋₆ alkoxy |
| C₁₋₆ alkyl | C₁₋₆ alkoxy |
| C₁₋₆ alkoxy | C₁₋₆ alkoxy |
| —C(O)OH | C₁₋₆ alkoxy |
| —S(O)₂—C₁₋₆ alkyl | C₁₋₆ alkoxy |
| Halo | —C(O)OH |
| —CN | —C(O)OH |
| —OH | —C(O)OH |
| —N(O)₂ | —C(O)OH |
| C₁₋₆ alkyl | —C(O)OH |
| C₁₋₆ alkoxy | —C(O)OH |
| —C(O)OH | —C(O)OH |
| —S(O)₂—C₁₋₆ alkyl | —C(O)OH |
| Halo | —S(O)₂—C₁₋₆ alkyl |
| —CN | —S(O)₂—C₁₋₆ alkyl |
| —OH | —S(O)₂—C₁₋₆ alkyl |
| —N(O)₂ | —S(O)₂—C₁₋₆ alkyl |
| C₁₋₆ alkyl | —S(O)₂—C₁₋₆ alkyl |
| C₁₋₆ alkoxy | —S(O)₂—C₁₋₆ alkyl |
| —C(O)OH | —S(O)₂—C₁₋₆ alkyl |
| —S(O)₂—C₁₋₆ alkyl | —S(O)₂—C₁₋₆ alkyl |

In an embodiment the compound is of one of the following Formulae:

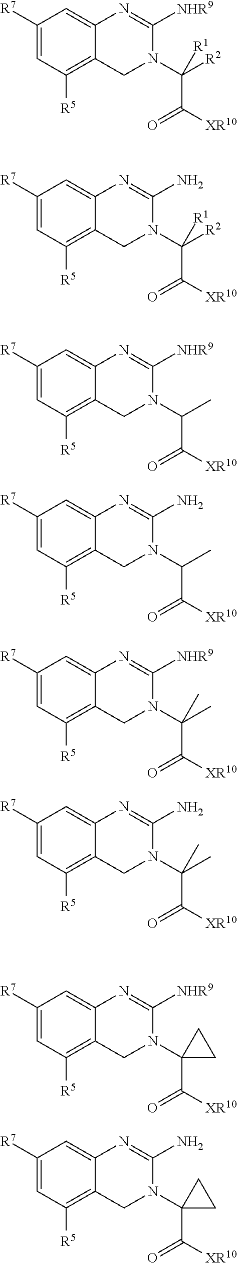

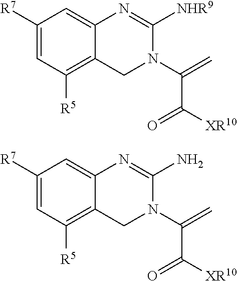

wherein R⁵ and R⁷ are each independently selected from R^{a} and R^{b};
   or, in each case, a pharmaceutically acceptable salt or solvate thereof.

With regard to each of said Formulae, R⁵ and R⁷ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁵ and R⁷ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment, R⁵ is R^{f} and R⁷ is R^{g}, wherein R^{f} and R^{g} are as defined in the Table above or are each halo.

In an embodiment the compound is of one of the following Formulae:

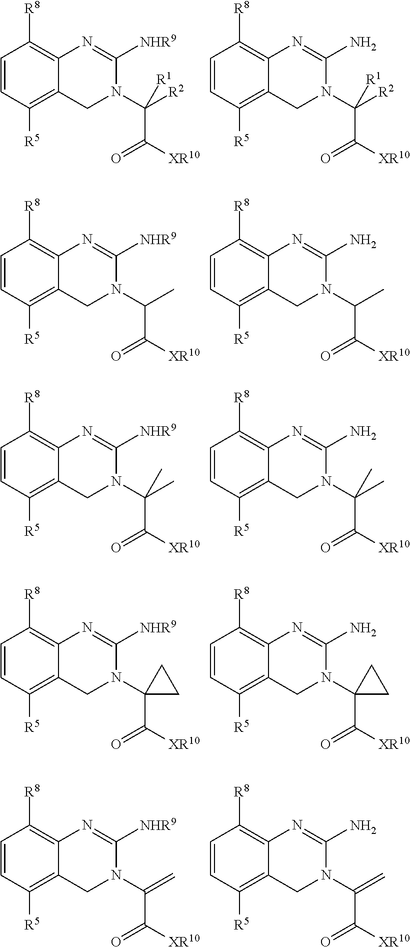

wherein R⁵ and R⁸ are each independently selected from R^{a} and R^{b};
   or, in each case, a pharmaceutically acceptable salt or solvate thereof.

With regard to each of said Formulae, R⁵ and R⁸ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁵ and R⁸ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment, R⁵ is R^{f} and R⁸ is R^{g}, wherein R^{f} and R^{g} are as defined in the Table above or are each halo.

In an embodiment the compound is of one of the following Formulae:

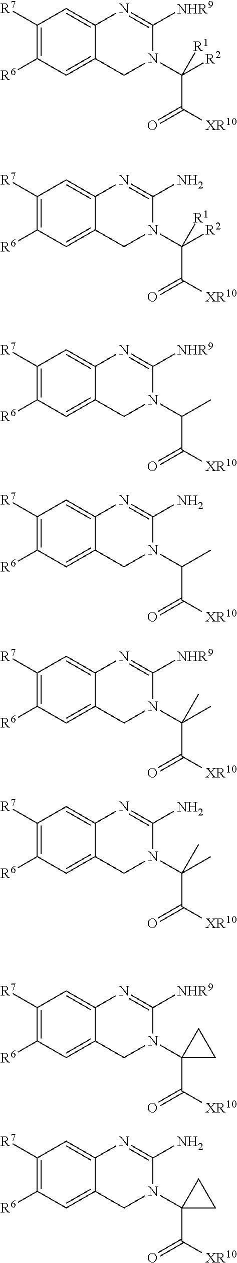

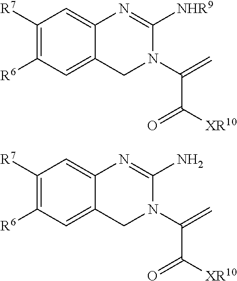

wherein R⁶ and R⁷ are each independently selected from R^{a} and R^{b};
   or, in each case, a pharmaceutically acceptable salt or solvate thereof.

With regard to each of said Formulae, R⁶ and R⁷ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁶ and R⁷ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment, R⁶ is R^{f} and R⁷ is R^{g}, wherein R^{f} and R^{g} are as defined in the Table above or are each halo.

In an embodiment the compound is of one of the following Formulae:

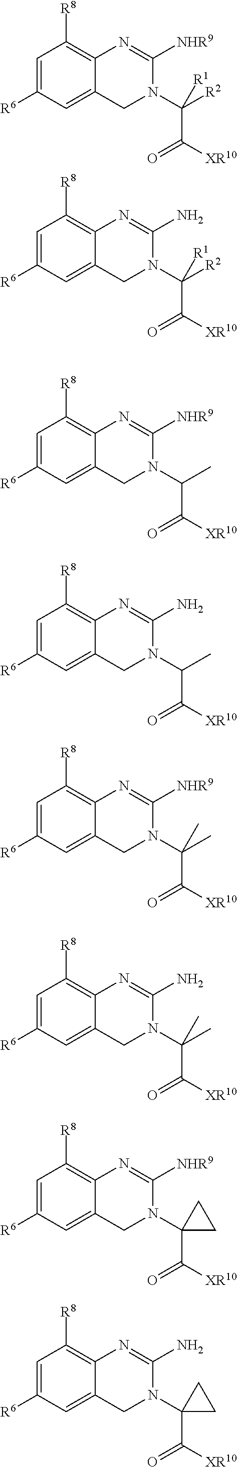

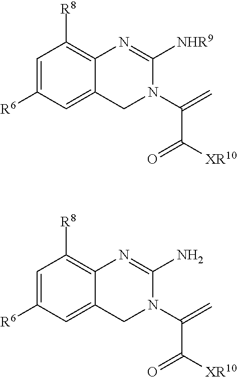

wherein R⁶ and R⁸ are each independently selected from R^{a} and R^{b};
   or, in each case, a pharmaceutically acceptable salt or solvate thereof.

With regard to each of said Formulae, R⁶ and R⁸ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁶ and R⁸ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment, R⁶ is R^{f} and R⁸ is R^{g}, wherein R^{f} and R^{g} are as defined in the Table above or are each halo.

In an embodiment the compound is of one of the following Formulae:

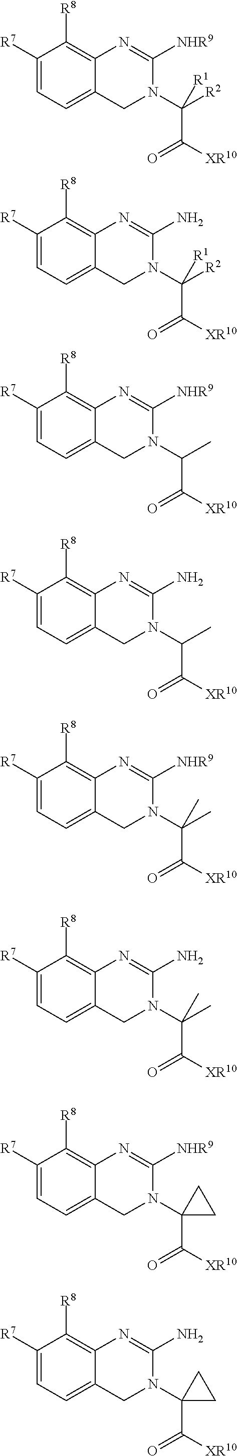

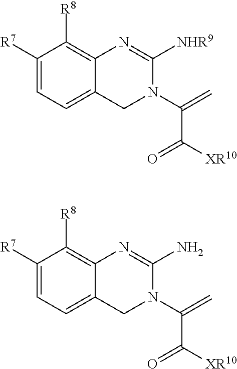

wherein R⁷ and R⁸ are each independently selected from R^{a} and R^{b};
   or, in each case, a pharmaceutically acceptable salt or solvate thereof.

With regard to each of said Formulae, R⁷ and R⁸ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁷ and R⁸ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment, R⁷ is R^{f} and R⁸ is R^{g}, wherein R^{f} and R^{g} are as defined in the Table above or are each halo.

In an embodiment, one of R⁵, R⁶, R⁷ and R⁸ is hydrogen, and the others are independently selected from R^{a} and R^{b}.

In an embodiment the compound is of one of the following Formulae:

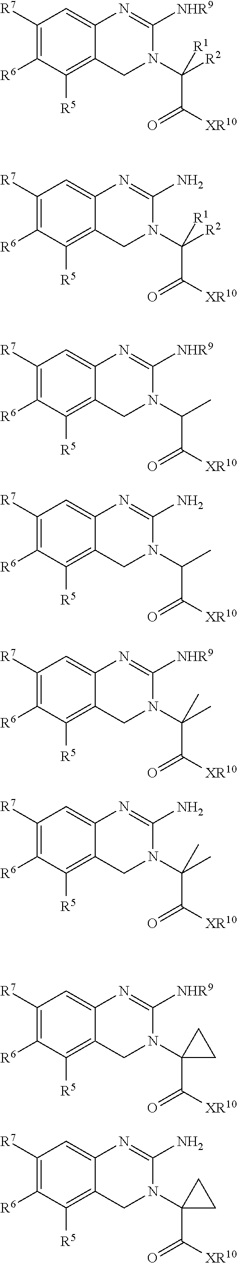

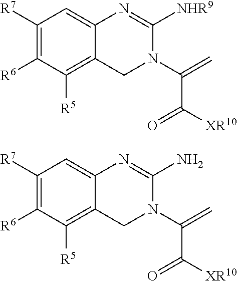

wherein R⁵, R⁶ and R⁷ are each independently selected from R^{a} and R^{b};
   or, in each case, a pharmaceutically acceptable salt or solvate thereof.

With regard to each of said Formulae, R⁵, R⁶ and R⁷ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁵, R⁶ and R⁷ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment the compound is of one of the following Formulae:

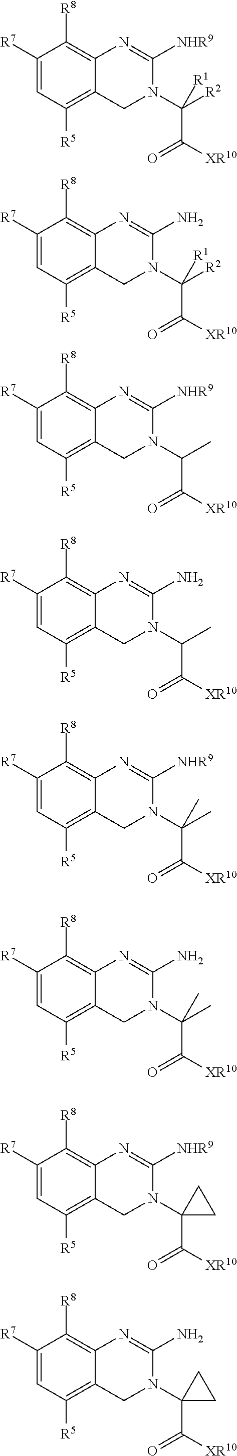

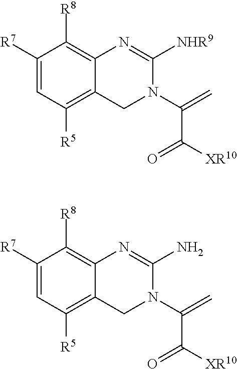

or, in each case, a pharmaceutically acceptable salt or solvate thereof.

With regard to each of said Formulae, R⁵, R⁷ and R⁸ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁵, R⁷ and R⁸ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment the compound is of one of the following Formulae:

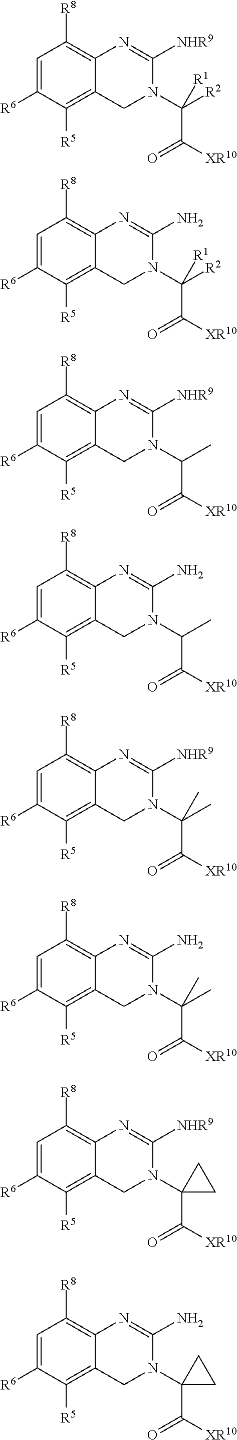

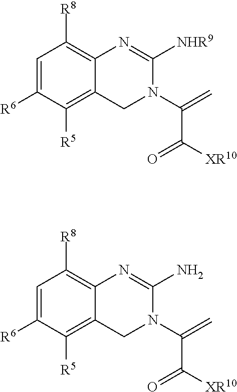

or, in each case, a pharmaceutically acceptable salt or solvate thereof.

With regard to each of said Formulae, R⁵, R⁶ and R⁸ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁵, R⁶ and R⁸ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl, dihalomethyl and methylsulphonyl.

In an embodiment the compound is of one of the following Formulae:

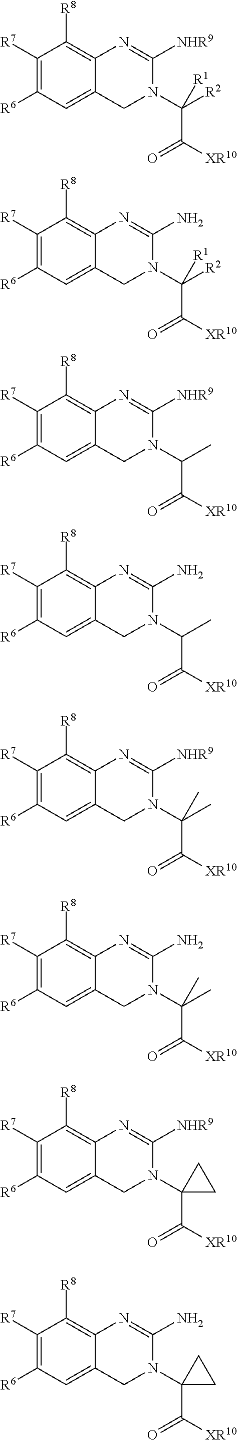

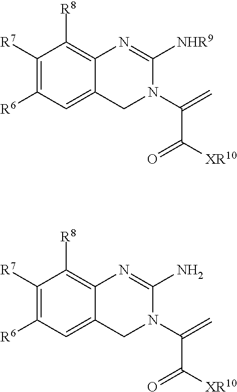

or, in each case, a pharmaceutically acceptable salt or solvate thereof.

With regard to each of said Formulae, R⁶, R⁷ and R⁸ may each be, for example, independently selected from R^{a} and R^{b}; wherein R^{a} is C₁₋₄ alkyl optionally substituted with 1, 2 or 3 R^{b}; and R^{b} is selected from fluoro, chloro, bromo, iodo, trifluoromethyl, cyano, nitro, —OR^{c}, —C(O)R^{c}, —C(O)OR^{c}, —OC(O)R^{c}, —S(O)₁R^{c} and —N(R^{c})R^{d}; wherein R^{c} and R^{d} are each independently hydrogen or C₁₋₄ alkyl optionally substituted with 1, 2 or 3 substituents independently selected from halo, cyano, amino, hydroxy, nitro and C₁₋₄ alkoxy. In an embodiment R⁶, R⁷ and R⁸ are each independently selected from fluoro, chloro, bromo, iodo, cyano, nitro, methyl, methoxy, trifluoromethyl, trifluoromethoxy, carboxylic acid, aminomethyl, fluoromethyl, chloromethyl, bromomethyl and methylsulphonyl.

In an embodiment, each of R⁵, R⁶, R⁷ and R⁸ is independently selected from R^{a} and R^{b}.

It has also been found that the individual enantiomers of the present compounds show efficacy. The present invention therefore also relates to both the resolved optical isomers of such compounds as well as mixtures of enantiomers. For the purposes of comparison of the compounds of the present invention with anagrelide, the correct comparison is that made with the PDE III inhibitory activity of the 3-hydroxy metabolite of anagrelide since this is the predominant component in plasma after anagrelide treatment.

Regarding the use of the compounds of the invention in humans, there is provided:

a pharmaceutical composition comprising a compound of formula (I), or a pharmaceutically acceptable salt or solvate thereof, together with a pharmaceutically acceptable diluent or carrier, which may be adapted for oral, parenteral or topical administration;
a compound of formula (I), or a pharmaceutically acceptable salt or solvate thereof, or a pharmaceutical composition containing any of the foregoing, for use as a medicament;
the use of a compound of formula (I), or a pharmaceutically acceptable salt or solvate thereof in the manufacture of a medicament for the treatment of a disease selected from: myeloprolific diseases and generalised thrombotic diseases; and
a method of treating a disease selected from: myeloprolific diseases and generalised thrombotic diseases in a human, which comprises treating said human with an effective amount of a compound of formula (I), or a pharmaceutically acceptable salt or solvate thereof, or with a pharmaceutical composition containing any of the foregoing.

The present invention also encompasses a method of treating a patient having essential thrombocythemia or other myelproliferative disease or thrombotic cardiovascular disease or high blood platelet count, which method comprises administering to the patient a therapeutically effective amount of a compound of the present invention.

Another embodiment of the present invention includes a method of reducing blood platelet count within a patient, which method comprises administering to the patient a therapeutically effective amount of a compound of the present invention.

The present invention encompasses providing the compounds of the present invention for the methods listed above, among others, wherein cardiotoxicity is reduced compared to using anagrelide.

The present invention also encompasses pharmaceutical compositions comprising a compound or pharmaceutically acceptable salt of a compound of the present invention and a pharmaceutically acceptable carrier.

Pharmaceutically acceptable salts of the compounds of Formula (I) include acid addition salts. Examples include hydrochloric and hydrobromide salts.

### DETAILED DESCRIPTION OF THE INVENTION

The present invention is directed to new prodrugs of substituted analogues of the established platelet lowering agent anagrelide. These compounds spontaneously ring close at pH's 7 and above to yield 3- or 5-substituted anagrelides that retain the anti-megakaryocytic properties (hence platelet lowering activity) of anagrelide but have reduced PDEIII inhibitory properties and hence lower potential for unwanted cardiovascular and anti-aggregatory side-effects.

Appropriate substitution at the 3-position of the anagrelide molecule effectively blocks the principal site of metabolism and thus precludes the formation of the highly potent PDEIII inhibitor 3-OH anagrelide. The 5-substituted analogues have the potential to indirectly sterically hinder metabolism at the preferred 3-position. These 3- or 5-substituted analogues of anagrelide also have the potential for improved pharmacokinetic characteristics since the 3-position in the anagrelide molecule is known to be the major site of metabolism which is the principal mechanism of drug clearance.

Use of the corresponding “open ring” prodrugs of these 3- or 5-substituted analogues could offer the added value of improved rates of dissolution and water solubility, allowing easier formulation. For example the aqueous solubility of anagrelide at pH 7 is <10 ug/ml. For ethyl-5,6-dichloro-3,4-dihydro-2-(1H)-iminoquinazoline-3-acetate HBr—an unsubstituted but representative example of these ring open prodrugs—the solubility is ˜5.5 mg/ml in distilled water.

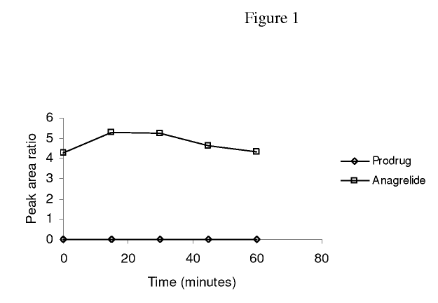

Such prodrugs are likely to be extremely rapidly and completely cyclised in plasma to the closed ring 3-alkylanagrelide analogues. For example the rapid and quantitative conversion of ethyl-5,6-dichloro-3,4-dihydro-2-(1H)-iminoquinazoline-3-acetate HBr—an unsubstituted but representative example of these ring open prodrugs—to anagrelide was demonstrated in human plasma using LC/MS-MS analytical techniques. Human plasma was spiked with anagrelide prodrug (final concentration 100 ng/mL). Immediately after mixing, and at 15, 30, 45 and 60 minutes afterward samples were analysed for anagrelide prodrug and anagrelide. Even at the first point of measurement no prodrug could be found demonstrating the rapid and complete conversion to anagrelide itself. FIG. 1 shows the levels of anagrelide prodrug, ethyl-5,6-dichloro-3,4-dihydro-2-(1H)-iminoquinazoline-3 acetate and anagrelide observed in samples of human plasma, incubated at room temperature over one hour.

The potential benefit of improved water solubility on the absorption of these open-ring analogues was shown in a comparative bioavailability study in the dog. Using the unsubstituted ethyl-5,6-dichloro-3,4-dihydro-2-(1H)-iminoquinazoline-3-acetate HBr as a model compound, a comparison was made of the systemic availability of anagrelide when given as this compound or as anagrelide itself in equimolar doses (7.7 & 6.1 mg/kg respectively). Examination of pharmacokinetic parameters for the prodrug showed an approximately 17-fold higher Cₘₐₓ, and a mean 16-fold higher AUC for anagrelide than when the drug itself was administered.

These results implied that the inherent absorption of anagrelide at this dose (6.1 mg/kg, albeit 200 fold above the clinical dose) was comparatively poor (<6.25%) since there was little evidence for marked changes in metabolism, the likely alternative explanation. The metabolite-to-drug exposure ratio after anagrelide was 1.5 compared to 0.9 after the prodrug.

This study (see tables below) also showed that there was also considerably less variability in Cₘₐₓ and AUC after the prodrug. For example Cₘₐₓ for anagrelide after the prodrug ranged from 170-418 ngmL⁻¹ (relative standard deviation, RSD, 26%) compared to 9.5 to 44.3 ngmL⁻¹ after anagrelide itself (RSD 62.5%). Similarly the AUC for anagrelide after the prodrug ranged from 367 to 1470 ng·hmL⁻¹ (RSD 34%) compared to 21.6 to 188 ng·hmL⁻¹ (RSD 71%) after anagrelide itself. The lesser variability was consistent with more efficient absorption. This study illustrated the potential benefits of the open-ring prodrugs to improve absorption.

TABLE 1
Pharmacokinetic parameters of anagrelide following a single oral
(capsule) administration of anagrelide or an ester open ring prodrug
of anagrelide to male dogs at equivalent molar doses

| | | | | | | |
|---|---|---|---|---|---|---|
| Dog ID | Cₘₐₓ | Tₘₐₓ | AUC₀₋ₜ | AUC_{infin} | k | t½ |
| number | (ng/mL) | (hours) | (ng · h/mL) | (ng · h/mL) | (hours⁻¹) | (hours) |

Anagrelide (6.1 mg/kg)

| | | | | | | |
|---|---|---|---|---|---|---|
| 1 | 15.7 | 16 | 141 | — | ^{e} | — |
| 3 | 14.8 | 1.5 | 42.0 | 42.3 ^{c} | 0.4459 ^{c} | 1.6 ^{c} |
| 11 | 25.0 | 2 | 188 | 193 ^{c} | 0.3119 ^{c} | 2.2 ^{c} |
| 23 | 9.50 | 1.5 | 21.6 | 23.1 ^{d} | 0.1953 ^{d} | 3.5 ^{d} |
| 29 | 44.3 | 1 | 88.9 | 89.3 | 0.3031 | 2.3 |
| Mean | 21.9 | 1.5 ^{b} | 96.3 | — | — | — |
| SD | 13.7 | | 68.9 | — | — | |

Ester prodrug of anagrelide (7.5 mg/kg)

| | | | | | | |
|---|---|---|---|---|---|---|
| 1 | 213 | 3 | 678 | 679 | 0.1969 | 3.5 |
| 3 ^{a} | 170 | 1 | 367 | 369 | 0.3071 | 2.3 |
| 11 | 418 | 4 | 1440 | 1440 ^{d} | 0.1789 ^{d} | 3.9 ^{d} |
| 23 | 334 | 3 | 951 | 952 | 0.4941 | 1.4 |
| 29 | 353 | 6 | 1470 | 1470 | 0.4857 | 1.4 |
| Mean | 330 | 3.5 ^{b} | 1130 | 1030 | 0.3922 | 1.8 ^{f} |
| SD | 86 | | 390 | 400 | 0.1692 | |

^{a} Animal vomited ca 1 hour post-dose, excluded from calculation of mean
^{b} Median
^{c} Estimate based on two data points only, therefore did not meet acceptance criteria, excluded from calculation of mean
^{d} Could not be estimated in accordance with all acceptance criteria, excluded from calculation of mean
^{e} Could not be estimated from the available data
^{f} Calculated as ln2/(mean rate constant)

TABLE 2
Pharmacokinetic parameters of 3-hydroxy anagrelide, following
a single oral (capsule) administration of anagrelide or an ester
prodrug of anagrelide to male dogs at equivalent molar doses

| | | | | | | |
|---|---|---|---|---|---|---|
| Dog ID | Cₘₐₓ | Tₘₐₓ | AUC₀₋ₜ | AUC_{infin} | k | t½ |
| number | (ng/mL) | (hours) | (ng · h/mL) | (ng · h/mL) | (hours⁻¹) | (hours) |

Anagrelide (6.1 mg/kg)

| | | | | | | |
|---|---|---|---|---|---|---|
| 1 | 14.1 | 16 | 131 | — | ^{d} | — |
| 3 | 18.0 | 1.5 | 64.6 | 65.0 | 0.2854 | 2.4 |
| 11 | 29.9 | 16 | 274 | — | ^{d} | — |
| 23 | 19.4 | 1.5 | 50.7 | 51.8 | 0.2314 | 3.0 |
| 29 | 43.0 | 1.5 | 122 | 123 | 0.2966 | 2.3 |
| Mean | 24.9 | 1.5 ^{b} | 128 | 79.9 | 0.2711 | 2.6 ^{e} |
| SD | 11.7 | | 89 | 37.9 | 0.0349 | |

Ester prodrug of anagrelide (7.5 mg/kg)

| | | | | | | |
|---|---|---|---|---|---|---|
| 1 | 185 | 3 | 564 | 566 | 0.1569 | 4.4 |
| 3 ^{a} | 106 | 1.5 | 303 | 303 | 0.2510 | 2.8 |
| 11 | 347 | 4 | 1280 | 1290 ^{c} | 0.1235 ^{c} | 5.6 ^{c} |
| 23 | 269 | 3 | 876 | 878 | 0.4425 | 1.6 |
| 29 | 241 | 6 | 1240 | 1240 | 0.3776 | 1.8 |
| Mean | 261 | 3.5 ^{b} | 990 | 895 | 0.3257 | 2.1 ^{e} |
| SD | 67 | | 337 | 337 | 0.1497 | |

^{a} Animal vomited ca 1 hour post-dose, excluded from calculation of mean
^{b} Median
^{c} Could not be estimated in accordance with all acceptance criteria (excluded from calculation of mean
^{d} Could not be estimated from the available data
^{e} Calculated as ln2/(mean rate constant)

For those 3- or 5-substituted anagrelide analogues which have a lower therapeutic potency (but not inherent activity) than anagrelide itself, a potentially higher absolute dose may be needed which could present problems for absorption. For example 3,3-dimethyl anagrelide (anti-megakaryocytic IC₅₀˜160 nM cf 27 nM for anagrelide) may need to be given at 6 times the current clinical dose of anagrelide. In this situation absorption may be less than complete and a prodrug may be needed to ensure efficient absorption from the GI tract.

It is to be understood that compounds of formula (I) may contain one or more asymmetric carbon atoms, thus compounds of the invention can exist as two or more stereoisomers.

Included within the scope of the present invention are all stereoisomers such as enantiomers and diastereomers, all geometric isomers and tautomeric forms of the compounds of formula (I), including compounds exhibiting more than one type of isomerism, and mixtures of one or more thereof.

Geometric isomers may be separated by conventional techniques well known to those skilled in the art, for example, by chromatography and fractional crystallisation.

Stereoisomers may be separated by conventional techniques known to those skilled in the art—see, for example, “Stereochemistry of Organic Compounds” by E L Eliel (Wiley, New York, 1994).

The compounds of Formula I can be prepared in an analogous manner to those described in U.S. Pat. No. 4,256,748 and U.S. Pat. No. 6,388,073. The disclosures of the synthetic procedures used in each of these documents is intended specifically to be incorporated into this disclosure and forms part of the disclosure of this invention. The contents are not presented here in full for the purposes of brevity but the skilled person is specifically directed to these documents.

A person skilled in the art will be aware of variations of, and alternatives to, the processes referred to in U.S. Pat. No. 4,256,748 which allow the individual compounds defined by formula (I) to be obtained having been now revealed as desirable targets. The present invention thus further encompasses methods of manufacturing a compound of the present invention to the extent that such processes produce novel intermediates and/or employ novel process features.

By way of illustration, and without limitation, a compound of the invention may be obtained according to the following reaction scheme (in which R is, for example, ethyl or other alkyl), using commercially available compounds:

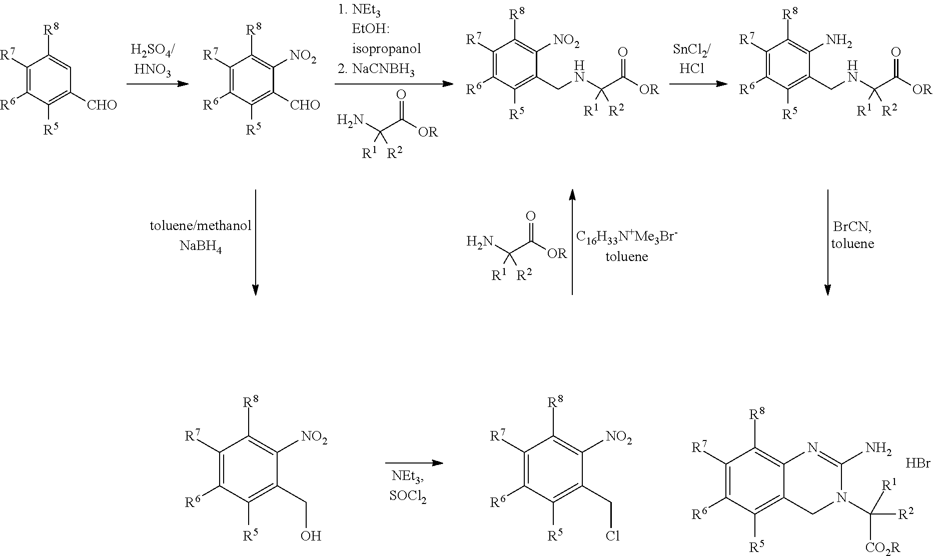

It will also be appreciated by a person skilled in the art that the compounds of the invention could be made by adaptation of the methods herein described and/or adaptation of methods known in the art, for example the art described herein, or using standard textbooks such as “Comprehensive Organic Transformations—A Guide to Functional Group Transformations”, R C Larock, Wiley-VCH (1999 or later editions), “March's Advanced Organic Chemistry—Reactions, Mechanisms and Structure”, M B Smith, J. March, Wiley, (5th edition or later) “Advanced Organic Chemistry, Part B, Reactions and Synthesis”, F A Carey, R J Sundberg, Kluwer Academic/Plenum Publications, (2001 or later editions), “Organic Synthesis—The Disconnection Approach”, S Warren (Wiley), (1982 or later editions), “Designing Organic Syntheses” S Warren (Wiley) (1983 or later editions), “Guidebook To Organic Synthesis” R K Mackie and D M Smith (Longman) (1982 or later editions), etc., and the references therein as a guide.

It will also be apparent to a person skilled in the art that sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and references therein.

### DEFINITIONS

Halo means a group selected from: fluoro, chloro, bromo or iodo.

The term “alkyl” as used herein as a group or a part of a group refers to a straight or branched hydrocarbon chain containing the specified number of carbon atoms. For example, C₁₋₁₀ alkyl means a straight or branched alkyl containing at least 1 and at most 10 carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl. A C₁₋₄ alkyl group is one embodiment, for example methyl, ethyl, n-propyl, isopropyl, n-butyl, isobutyl or t-butyl.

The term “cycloalkyl” as used herein refers to a non-aromatic monocyclic hydrocarbon ring of 3 to 8 carbon atoms such as, for example, but not limited to, cyclopropyl, cyclobutyl, cyclopentyl, cyclohexyl or cycloheptyl.

The term “spirocyclic” as used herein refers to a ring system joined to a second ring system at one carbon atom.

The term “alkoxy” as used herein refers to a straight or branched hydrocarbon chain group containing oxygen and the specified number of carbon atoms. For example, C₁₋₆ alkoxy means a straight or branched alkoxy containing at least 1 and at most 6 carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄ alkoxy group is one embodiment, for example methoxy, ethoxy, propoxy, prop-2-oxy, butoxy, but-2-oxy or 2-methylprop-2-oxy.

The term “hydroxyalkyl” as used herein as a group refers to a straight or branched hydrocarbon chain containing the specified number of carbon atoms, which is substituted by 1-3 hydroxyl groups. For example, C₁₋₄ hydroxyalkyl means a straight or branched alkyl chain containing from 1 to 4 carbon atoms and at least one hydroxyl group; examples of such group include hydroxymethyl, hydroxyethyl, hydroxypropyl, hydroxyisopropyl, hydroxybutyl and the like.

The term “alkenyl” as used herein as a group or a part of a group refers to a straight or branched hydrocarbon chain containing the specified number of carbon atoms and containing at least one double bond. For example, the term “C₂₋₆ alkenyl” means a straight or branched alkenyl containing at least 2 and at most 6 carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl. It will be appreciated that in groups of the form —O—C₂₋₆ alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₂₋₆ alkynyl” means a straight or branched alkynyl containing at least 2 and at most 6 carbon atoms and containing at least one triple bond. Examples of “alkynyl” as used herein include, but are not limited to, ethynyl, 2-propynyl, 3-butynyl, 2-butynyl, 2-pentynyl, 3-pentynyl, 3-methyl-2-butynyl, 3-methylbut-2-ynyl, 3-hexynyl and 1,1-dimethylbut-2-ynyl. It will be appreciated that in groups of the form —O—C₂₋₆ alkynyl, the triple bond is preferably not adjacent to the oxygen. The term “halo” refers to halogens such as fluorine, chlorine, bromine or iodine atoms.

The term “sulfide” refers to a radical of Rₐ—S—R_{b}, wherein a sulfur atom is covalently attached to two hydrocarbon chains, Rₐ and R_{b}, wherein the two hydrocarbon chains may be, for example, but not limited to, any discussed above.

The compounds of the invention, i.e. those of formula (I), when cyclised may possess antimegakaryocytic activity in humans. Such activity may be assessed using a well established model. Assessment of the in vitro anti-megakaryocytic activity—and potentially therefore the platelet lowering capability—of the anagrelide prodrugs can be determined using the model of megakaryocytopoiesis (Cohen-Solal et al., Thromb. Haemost. 1997, 78:37-41 and Cramer et al., Blood, 1997, 89:2336-46). This involves examining the differentiation of human CD34⁺ stem cells into megakaryocytes which ultimately give rise to blood platelets.

The compounds of the invention may be particularly useful in the treatment of myeloproliferative diseases. The compounds may also find utility in the treatment of generalised thrombotic diseases.

It is to be appreciated that references to treatment include prophylaxis as well as the alleviation and/or cure of established symptoms of a condition. “Treating” or “treatment” of a state, disorder or condition includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

Myeloproliferative diseases which may be treatable with the compounds of the present invention include: essential thrombocythemia, polycythema vera, chronic idiopathic myelofibrosis, chronic myeloid leukaemia with residual thrombocytosis, reactive thrombocytosis immediately preceding a surgical procedures, as an immediate or post operative preventative measures to minimise the risk of thrombus formation during or post surgery.

Thrombotic cardiovascular diseases (TCVD) (i.e. patients at increased generalised thrombotic risk) which may be treatable with the compounds of the present invention include: myocardial infarct (heart attack) thrombotic stroke, patients having undergone coronary stent placement.

The compounds of the present invention may also find utility in indicated for the reduction of atherothrombotic events as follows: recent MI, recent stroke or established peripheral arterial disease, acute coronary syndrome (unstable angina/non-Qwave MI), cardiovascular death, MI, stroke, and refractory ischemia.

Compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

They may be administered alone or in combination with one or more other compounds of the invention or in combination with one or more other drugs. Generally, they will be administered as a formulation in association with one or more pharmaceutically acceptable excipients. Pharmaceutically acceptable excipients include one or more of: anti-oxidants, colourants, flavouring agents, preservatives and taste-masking agents.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995). The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The methods by which the compounds may be administered include oral administration by capsule, bolus, tablet, powders, lozenges, chews, multi and nanoparticulates, gels, solid solution, films, sprays, or liquid formulation. Liquid forms include suspensions, solutions, and syrups. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid preparation, for example, from a sachet.

The compounds may also be administered topically to the skin or mucosa, that is dermally or transdermally. Typical formulations for this purpose include pour-on solutions, sprays, powder formulations, gels, hydrogels, lotions, creams, ointments, films and patches, and implants.

The compounds can also be administered parenterally, or by injection directly into the blood stream, muscle or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Formulations may be immediate and/or modified controlled release. Controlled release formulations include Modified release formulations include: delayed-, sustained-, and pulsed-release.

### Dosages

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

In general however a suitable dose will be in the range of from about 0.001 to about 50 mg/kg of body weight per day, in a further embodiment, of from about 0.001 to about 5 mg/kg of body weight per day; in a further embodiment of from about 0.001 to about 0.5 mg/kg of body weight per day and in yet a further embodiment of from about 0.001 to about 0.1 mg/kg of body weight per day. In further embodiments, the ranges can be of from about 0.1 to about 750 mg/kg of body weight per day, in the range of 0.5 to 60 mg/kg/day, and in the range of 1 to 20 mg/kg/day.

The desired dose may conveniently be presented in a single dose or as divided doses administered at appropriate intervals, for example as one, two, three, four or more doses per day. If the compounds are administered transdermally or in extended release form, the compounds could be dosed once a day or less.

The compound is conveniently administered in unit dosage form; for example containing 0.1 to 50 mg, conveniently 0.1 to 5 mg, most conveniently 0.1 to 5 mg of active ingredient per unit dosage form. In yet a further embodiment, the compound can conveniently administered in unit dosage form; for example containing 10 to 1500 mg, 20 to 1000 mg, or 50 to 700 mg of active ingredient per unit dosage form.

1. A compound of Formula (I) or a pharmaceutically acceptable salt or solvate thereof 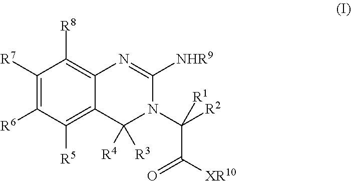
wherein:
R¹, R², R³ and R⁴ independently represent hydrogen or a blocking group which functions to prevent metabolic reaction either directly or indirectly at the carbon atom to which R¹ and R² are attached;
or R¹ and R², and/or R³ and R⁴ together with the carbon to which they are attached form a blocking group which functions to prevent metabolic reaction at the carbon atom to which R¹ and R² are attached, the remainder of groups R¹ to R⁴ being hydrogen;
R⁵, R⁶, R⁷ and R⁸ are each independently selected from hydrogen, R^{a} and R¹);
R⁹ is H or C₁₋₆ alkyl;
R¹⁰ is selected from the group comprising: hydrogen; C₁₋₆ alkyl, C₂₋₆ alkenyl, C₂₋₆ alkynyl and C₃₋₈ cycloalkyl wherein each of the foregoing groups may be optionally substituted by 1 to 5 groups chosen independently from the group comprising: halo, hydroxyl, cyano, nitro, C₁₋₄ alkylsulphonyl and COOH; or R¹⁰ is a pharmaceutically acceptable cation;
X is O or S;
R^{a} is selected from C₁₋₆ alkyl and C₂₋₆ alkenyl, either of which is optionally substituted with 1, 2, 3, 4 or 5 R^{b};
R^{b} is selected from halo, trifluoromethyl, cyano, nitro, —OR^{c}, —C(O)R^{c}, —C(O)OR^{c}, —OC(O)R^{c}, —S(O)₁R^{c}, —N(R^{c})R^{d}, —C(O)N(R^{c})R^{d}, —N(R^{c})C(O)R^{d}, —S(O)₁N(R^{c})R^{d} and —N(R^{c})S(O)₁R^{d};
R^{c} and R^{d} are each independently hydrogen or R^{e};
R^{e} is selected from C₁₋₆ alkyl and C₂₋₆ alkenyl, either of which is optionally substituted with 1, 2, 3, 4 or 5 substituents independently selected from halo, cyano, amino, hydroxy, nitro and C₁₋₆ alkoxy; and
1 is 0, 1 or 2;
and wherein each of the following provisos applies:
(i) R¹, R², R³ and R⁴ are not all hydrogen; and
(ii) when R⁵ and R⁶ are each halo, then R⁷ and R⁸ are not both selected from H, halo, cyano, C₁₋₆ alkyl, C₁₋₆ haloalkyl, C₁₋₆ alkoxy and C₁₋₆ haloalkoxy.

2. A compound of claim 1, wherein
R¹ and R², are independently selected from the group comprising: H; halo; cyano; C₁₋₆ alkyl, C₂₋₆ alkenyl, C₂₋₆ alkynyl, C₃₋₄ cycloalkyl wherein said alkyl, alkenyl, alkynyl or cycloalkyl groups may be optionally substituted by 1 to 5 groups chosen independently from the group comprising: halo, hydroxyl, cyano, nitro, C₁₋₄ alkylsulphonyl and COOH; C₁₋₆ hydroxyalkyl; carboxyalkyl; and sulphide;
or wherein R₁ and R₂ together with the carbon to which they are attached form a C₃₋₈ carbocyclic ring may be optionally substituted by 1 to 5 groups chosen independently from the group comprising: halo, hydroxyl, cyano, nitro, C₁₋₄ haloalkyl, C₁₋₄ alkylsulphonyl and COOH;
or wherein R₁ and R₂ together with the carbon to which they are attached represent a C₂₋₆ alkenyl or C₂₋₆ alkynyl group bound through a double bond to the ring to which it is attached and being optionally substituted by one to three groups independently selected from the group comprising: halo, hydroxyl, cyano, C₁₋₄ haloalkyl and COOH, provided always that one of R¹ and R² is not hydroxyl when the other is methyl.

3. A compound according to claim 1, wherein R³ and R⁴ are independently selected from the group comprising: H; halo; cyano; C₁₋₆ alkyl, C₂₋₆ alkenyl, C₂₋₆ alkynyl, C₃₋₈ cycloalkyl wherein said alkyl, alkenyl, alkynyl or cycloalkyl groups may be optionally substituted by 1 to 5 groups chosen independently from the group comprising: halo, hydroxyl, cyano, nitro, C₁₋₄ alkylsulphonyl and COOH; C₁₋₆ hydroxyalkyl; C₁₋₆ carboxyalkyl; and sulphide;
or R³ and R⁴ together with the carbon to which they are attached form a C₃₋₄ carbocyclic ring may be optionally substituted by 1 to 5 groups chosen independently from the group comprising: halo, hydroxyl, cyano, nitro, C₁₋₄ haloalkyl, C₁₋₄ alkylsulphonyl and COOH;
or R³ and R⁴ together represent a C₂₋₆ alkenyl or C₂₋₆ alkynyl group bound through a double bond to the ring to which it is attached and being optionally substituted by one to three groups independently selected from the group comprising: halo, hydroxyl, cyano, C₁₋₄ haloalkyl and COOH.

4. A compound as claimed in claim 1, wherein R¹ is an optionally substituted C₁₋₄ alkyl or C₃₋₄ cycloalkyl group.

5. A compound as claimed in claim 1, wherein R² is an optionally substituted C₁₋₄ alkyl or C₃₋₄ cycloalkyl group.

6. A compound as claimed in claim 1, wherein R¹ is methyl, cyclopropyl, CF₃ or CHF₂.

7. A compound as claimed in claim 1, wherein R² is methyl, cyclopropyl, CF₃ or CHF₂.

8. A compound as claimed in claim 1, wherein R¹ and R² together form an optionally substituted C₃₋₄ cycloalkyl group.

9. A compound as claimed in claim 1, wherein R³ is H or C₁₋₆ alkyl.

10. A compound as claimed in claim 1, wherein R⁴ is H or C₁₋₆ alkyl.

11. A compound as claimed in claim 1, wherein two of R⁵, R⁶, R⁷ and R⁸ are hydrogen, and the other two are independently selected from R^{a} and R^{b}.

12. A compound as claimed in claim 11, wherein R⁷ and R⁸ are each hydrogen.

13. A compound according to claim 1, wherein three of R⁵, R⁶, R⁷ and R⁸ are hydrogen, and the other is selected from R^{a} and R^{b}.

14. A compound according to claim 13, wherein R⁷ and R⁸ are each hydrogen.

15. A compound as claimed in claim 1, wherein R⁹ is H.

16. A compound as claimed in claim 1, wherein R⁹ is methyl.

17. A compound as claimed in claim 1, wherein R¹⁰ is C₁₋₆ alkyl.

18. A compound as claimed in claim 1, wherein R^{a} is C₁₋₆ alkyl optionally substituted with 1, 2, 3, 4 or 5 R^{b}; and R^{b} is selected from halo, cyano, nitro, —OH, C₁₋₆ alkoxy, —C(O)OH and —S(O)₂—C₁₋₆ alkyl.

19. A pharmaceutical composition comprising a compound of formula (I) as defined in claim 1, or a pharmaceutically acceptable salt or solvate thereof, together with a pharmaceutically acceptable diluent or carrier, which may be adapted for oral, parenteral or topical administration.

20. A compound of formula (I) as defined in claim 1, or a pharmaceutically acceptable salt or solvate thereof, or a pharmaceutical composition containing any of the foregoing, for use as a medicament.

21. The use of a compound of formula (I) as defined in claim 1, or a pharmaceutically acceptable salt or solvate thereof in the manufacture of a medicament for the treatment of a disease selected from: myeloprolific diseases and generalised thrombotic diseases.

22. A method of treating a disease selected from: myeloproliferative diseases and generalised thrombotic diseases in a human, which comprises treating said human with an effective amount of a compound of formula (I) as defined in claim 1, or a pharmaceutically acceptable salt or solvate thereof, or with a pharmaceutical composition containing any of the foregoing.

23. Use of a compound of formula (I) as defined in claim 1 for the reduction of platelet count.
